(19) United States
(12) Patent Application Publication
(10) Pub. No.: US 2016/0183865 A1
(43) Pub. Date: Jun. 30, 2016

(54) METHOD FOR DIAGNOSING CRANIAL BONE DISPLACEMENT

(71) Applicant: Andrii Hennadiiovych MURAEV, Obuhiv (UA)

(72) Inventor: Andrii Hennadiiovych MURAEV, Obuhiv Kyivska obl. (UA)

(21) Appl. No.: 14/910,006

(22) PCT Filed: Jul. 24, 2014
(86) PCT No.: PCT/UA2014/000078

§ 371 Date: Feb. 4, 2016

(30) Foreign Application Priority Data
Aug. 14, 2013 (UA) U 2013 10089

(51) Int. Cl. A61B 5/00; A61B 5/107

(52) U.S. Cl. CPC A61B 5/4504; A61B 5/1072; A61B 5/0059

(57) ABSTRACT

A method for diagnosing cranial one displacement by determining the location of characteristic points on the skull such as an occipital point situated on the protuberance of the occipital bone at the centre of the occiput, a frontal point situated at the intersection of the external base of the skull and the sagittal suture of the frontal bone, a right-hand temporal point situated at the intersection of the external base of the skull and a vertical line from the right-hand mandibular notch, a left-hand temporal point situated at the intersection of the external base of the skull and a vertical line from the left-hand mandibular notch, and a diagnostic point situated at the intersection of a line passing through the occipital and frontal points and a line passing through the right-hand temporal point and the left-hand temporal point; the distance between the occipital and frontal points is measured along the right-hand outer part of the head and, separately, along the left-hand outer part of the head, the distance between the right-hand and left-hand temporal points is measured across the frontal bone, and the distance between said points is measured across the occipital bone, the distances between the occipital point and the diagnostic point and between the frontal point and the diagnostic point are measured across the upper part of the skull, and the distances between the left-hand temporal point and the diagnostic point and between the right-hand temporal point and the diagnostic point are measured, the values of the corresponding results obtained are compared and cranial bone displacement is determined according to the deviation of said values from zero.

### CROSS-REFERENCE TO RELATED APPLICATIONS

This application is a National stage application of PCT application PCT/UA2014/000078 fled on Jul. 24, 2014, claims priority to Ukrainian application 2013 10089 filed on Aug. 14, 2013.

### FIELD OF THE INVENTION

The invention relates to the field of medicine, namely, to the research methods of the form, structure, body size, or its part, the cranium in particular, for diagnostic purposes. The invention can be used in early preclinical, clinical recognition and treatment of the post-traumatic syndrome, brain concussion, brain contusion, cranium deformation, traumatic brain injury consequences, and for rehabilitation purposes after head injury.

### BACKGROUND

It is known that cranium bones don't fully knit over time, but instead form so called sutures, which in their turn form cranium articulations, which have certain degree of mobility. Hits to the head and concussions from falls, punches and strikes in boxing and other martial arts may lead to the cranium bones displacement. The consequences of the cranium bones displacement may be quite serious—up to suspended cerebral fluid flow and cerebral aqueduct dislocation as the liquid itself can't be compressed. Abnormal mobility of cranium bones movement may cause compression of the part of the brain, poor blood circulation, nerve irritation, and/or vision and hearing impairments.

There is a known method of integrated manual diagnostics, which contemplates doctor's fingers being placed on the bone landmarks of patient's head and neck, active head turns performed by the patient, while maintaining the vertical axis of the spine which detects the pathology or its absence with subsequent correction (Patent #RU 2248195, IPC7 A61N 1/00, the date of publ. May 20, 2010).

The drawback of the above method is that if the patient is in pain, the protracted examination which includes active head turns may exhaust the patient and cause discomfort.

There is a another known diagnostic method for detection of the cranium bones displacement via determining the placement of the characteristic points on the cranium, that contemplates the placement of doctor's fingers on bone landmarks of the patient's head, palpates the cranium bones and conducts the physiological tests to determine the cranium bones displacement or its absence and the type of displacement by placing the left hand under the patient's occipital bone and fixating the thumb, forefinger, middle and ring fingers of the left hand on the on the occipital bone and fixating the thumb, forefinger middle and ring fingers of the right hand on the wing of the sphenoid bone (Patent # RU 2389463 C1, IPC A61N 1/00 (January 2006), publ. May 20, 2008). To determine the mobility of the bones the distance between the occipital and the sphenoid is reduced by simulating the periodical pressure.

The drawback of the above method is the fact that the exact degree of cranium bones displacement can't be determined, as doctor does the examination based on his/her sensation, which requires long-term special training in the field of manual therapy.

### SUMMARY

The objective of the present invention is improvement of the cranium bones displacement diagnostic method, such that the new method would reveal the possible deviation of the points on the cranium from the normal position and symmetry relative to both right and left sides of the cranium, on the one side, and relative to frontal and occipital sides of the cranium on the other side, while detecting the degree and direction of the displacement by determining the characteristic points location on the cranium and measuring the scull circumference.

The problem as specified above is solved by using the method where such characteristic points location on the scull are determined—occipital point located on the occipital bone protuberance in the center of occiput, frontal point located at the intersection of the outer base of the cranium and the sagital suture of the frontal bone, right-sided temporal point located at the intersection of the external base of the cranium and the vertical line of the mandibular fosse on the right and left temporal point located at the intersection of the external base of the cranium and the vertical line of the mandibular fossa on the left, and a diagnostic point that is on the intersection of the line passing through the occipital and frontal point, with the line passing through the right-temporal lobe and a left-temporal point. The distance is measured along the outer part of the cranium between the occipital and frontal points to the right and separately to the left. The distance is also measured between right and left temporal points through the frontal bone, and the distance between them through the occipital bone. The distance is measured from the top of the cranium between the occipital and diagnostic points and the distance between the frontal and diagnostic points, and measure the distance between the left temporal and diagnostic points between the right temporal and diagnostic points. Then the obtained corresponding distance values are compared for the left and right sides, the difference between the values is determined. If the difference between the values differs from zero; the displacement of the cranium bones is stated.

In some embodiments the distances between the featured points are measured by well-known means of 3D scanner or video photographic equipment or wave radiation equipment, or mechanical tools that do not stretch, such as a thread or a tape measure.

Determination of the characteristic points and the measurement of the distances between them:
between the occipital and frontal points on the right,
between the occipital and frontal points on the left,
between right and left temporal points through the frontal bone,
between right and left temporal points through the occipital bone,
between the occipital and diagnostic points through the top part of the cranium,
between the frontal and diagnostic points through the top part of the cranium,
between the left temporal and diagnostic points and
between the right temporal and diagnostic points
makes it possible to find out the digital values of the specified distances on the cranium, which allows to identify the specific displacement of the relevant bones of the cranium with respect to the horizontal and vertical directions and come up with the treatment plan.

### BRIEF DESCRIPTION OF DRAWINGS

The content of the invention is explained by drawings, which are shown schematically:

FIG. 1—the location of the characteristic point

FIG. 2—The location of the frontal characteristic point

FIG. 3—The location of a left temporal characteristic point,

FIG. 4—location of the right temporal characteristic point

FIG. 5—the location of the diagnostic characteristic point.

### DETAILED DESCRIPTION OF THE PREFERRED EMBODIMENT

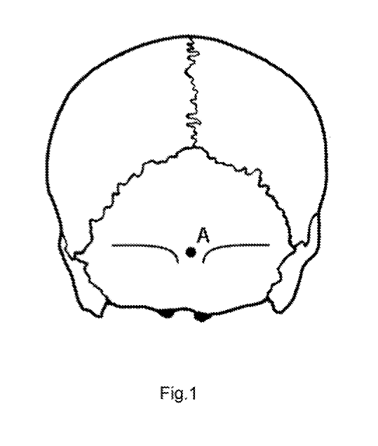

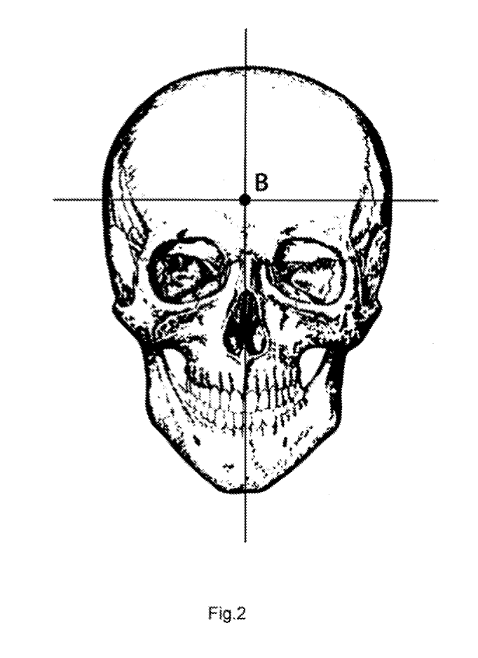

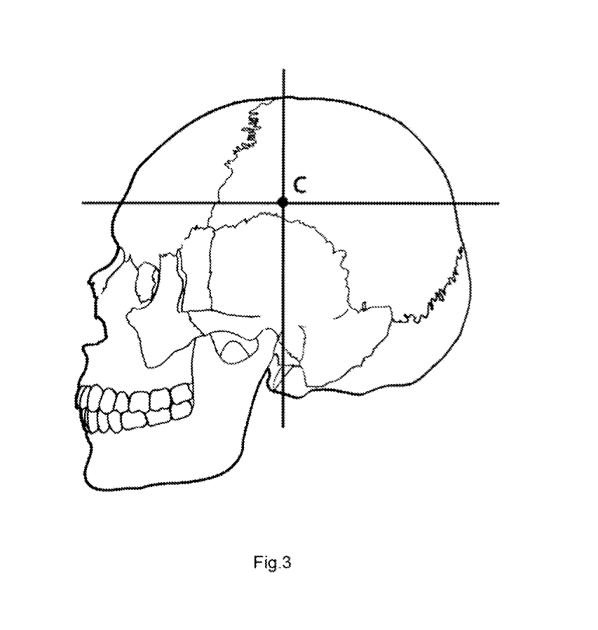

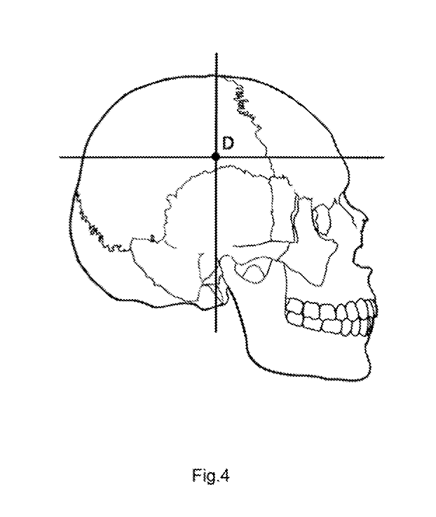

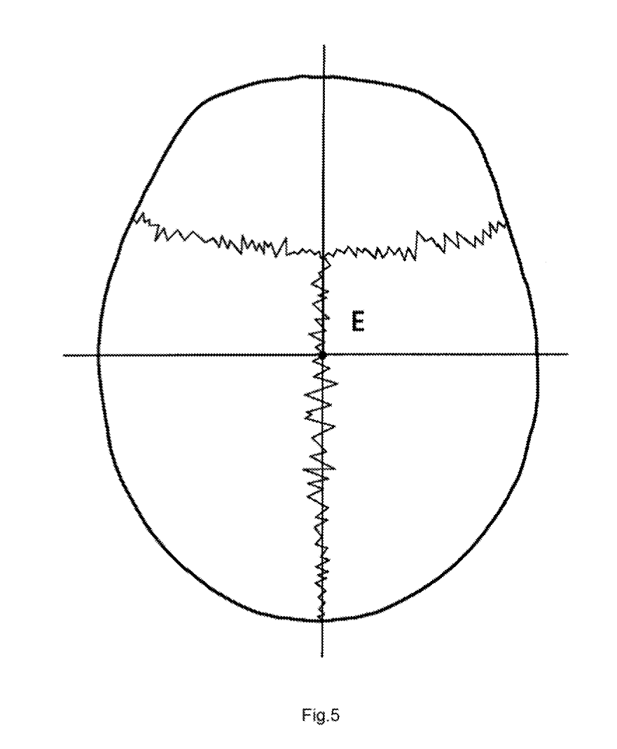

The core of the A. G. Muraev method for diagnosing cranium displacement is to determine the location of the characteristic points on the cranium. These characteristic points are the occipital point A, located on the protuberance of the occipital bone in the middle of the occiput (see. FIG. 1), the frontal point B, located at the intersection of the outer base of the cranium and the sagittal suture of the frontal bone (see. FIG. 2), a left temporal point C, located at the intersection of the outer base of the cranium and the vertical line from mandibular fossa on the left (see. FIG. 3) and the right-temporal point D, located at the intersection of the outer base of the cranium and the vertical line from mandibular fosse on the right (see. FIG. 4). Diagnostic point E (see. FIG. 5) is determined at the intersection of a line passing through the frontal and occipital A and frontal B points and the line passing through the right temporal C and left temporal D points.

After that, by means of 3D scanner, or video-photographic equipment, or wave of radiation equipment, or tools that do not stretch, such as a thread or tape measure, the size of the outer part of the cranium is measured:
between the occipital A and frontal B points on the right,
between the occipital A and frontal B points on the left,
between left C and right temporal points through the frontal bone,
between left C and right D temporal points through the occipital bone,
between the occipital A and diagnostic E points through the top of the cranium,
between the frontal B and diagnostic E points through the top of the cranium,
between the left temporal C and the diagnostic E points and
between right temporal and diagnostic E points.

The measured values are compared and the cranium bones displacement is determined by its deviation from zero.

The AB distance is compared both on the left and the right side.

If AB right>AB left, it means that the cranium bones are displaced to the right, If AB right<AB left it means that the cranium bones are displaced to the left. By subtracting the denoted values, the degree of the cranium bones displacement can be determined.

If AB right=AB left, it means that the scull bones displacement is absent.

The degree of cranium bones displacement, notably the deviations from symmetry, can be calculated by comparing the values of CD values through the frontal and occipital part of the cranium.

Also, the values of AE and EB, EC and ED are compared.

Once all values are analyzed, it is possible to present a complete picture of the location of all cranium bones of the patient, the displacement direction and its value, which allows to make with the consistent treatment plan.

1. A method of detection of a bone misalignment of a cranium, comprising:
determining positions of characteristic points on the cranium:
an occipital point located on an occipital bone protuberance in a center of an occiput,
a frontal point located at an intersection of an outer base of the cranium and a sagittal suture of a frontal bone,
a right-sided temporal point located at an intersection of an external base of the cranium and a vertical line of a mandibular fossa on a right side and
a left-sided temporal point located at an intersection of the external base of the cranium and a vertical line of the mandibular fossa on a left side, and
a diagnostic point that is on an intersection of a line passing through the occipital and frontal points with a line passing through the right-sided temporal point and the left-sided temporal point,
measuring distances on an outer part of the cranium between the following points:
the occipital and the frontal points to the right and to the left separately;
the right-sided and the left-sided temporal points through the frontal bone;
the right-sided and the left-sided temporal points through an occipital bone;
the occipital and the diagnostic points via a top of the cranium; and
the frontal and the diagnostic points;
the left-sided temporal and the diagnostic points;
the right-sided temporal and the diagnostic points; and
comparing values of corresponding left and right distances;
determining a difference between values of corresponding left and right distances; and
if the difference of the values is not equal to zero making a conclusion on a presence of the misalignment of the corresponding cranium bones.

2. The method of claims 1, wherein the distances between the above-mentioned points are measured by means of a 3D scanner.

3. The method of claims 1, wherein the distances between the above-mentioned points are measured by using video photographic equipment.

4. The method of claims 1, wherein the distances between the above-mentioned points are measured by electromagnetic radiation equipment.

5. The method of claims 1, where the distances between the above-mentioned points are measured by mechanical tools that do not stretch, including a non-elastic thread or a tape.
